(19) United States
(12) Patent Application Publication
(10) Pub. No.: US 2026/0314943 A1
(43) Pub. Date: Oct. 15, 2026

(54) BLOOD PRESSURE MEASUREMENT METHOD AND ELECTRONIC DEVICE SUPPORTING THE SAME

(71) Applicant: SAMSUNG ELECTRONICS CO., LTD., Suwon-si (KR)

(72) Inventors: Changsoon PARK, Suwon-si (KR); Daegeun JANG, Suwon-si (KR); Sunok JUNG, Suwon-si (KR); Jinwoo SEO, Suwon-si (KR); Sunghwan CHO, Suwon-si (KR)

(21) Appl. No.: 19/694,044

(22) Filed: Jun. 1, 2026

Related U.S. Application Data
(63) Continuation of application No. PCT/KR2024/016183, filed on Oct. 23, 2024.

(30) Foreign Application Priority Data
Dec. 11, 2023 (KR) 1020230178263
Dec. 19, 2023 (KR) 1020230186021

(51) Int. Cl. A61B 5/0205; A61B 5/00; A61B 5/021; A61B 5/11

(52) U.S. Cl. CPC A61B 5/0205; A61B 5/681; A61B 5/742; A61B 5/02141; A61B 5/1116

(57) ABSTRACT

An electronic device includes: a first sensor which collects first information, a second sensor which collects second information related to the orientation of the electronic device, and a third sensor which collects third information related to the temperature of a measurement area. In response to determining the signal quality of a biometric signal represented by the first information is lower than a specified level, the electronic device acquires, on the basis of the second information and the third information, a first result indicating whether the orientation of the electronic device corresponds to a designated reference orientation, and a second result indicating whether the temperature of the measurement area corresponds to a designated reference temperature. The electronic device selectively provides a first guide related to the orientation of the electronic device or a second guide related to temperature of the measurement area, based on the first result and the second result.

This application is a continuation application, claiming priority under § 365(c), of International Application No. PCT/KR2024/016183, filed Oct. 23, 2024, which is based on and claims the benefit of Korean patent application No. 10-2023-0178263, filed on Dec. 11, 2023, and Korean patent application No. 10-2023-0186021, filed on Dec. 19, 2023, the disclosures of which are incorporated by reference herein in their entireties.

### BACKGROUND

### Field

Embodiments disclosed in the disclosure relate to a blood pressure measurement method and an electronic device supporting the same.

### Description of the Related Art

As electronic devices have gradually become smaller while being able to perform the same function or more various functions, electronic devices have been developed in forms which are easy to carry. In general, such electronic devices are stored and carried in a pocket of a user or the like, but may be worn on the wrist of the user or may be worn on a head portion or an arm of the body of the user.

In some cases, an electronic device may include a mounted biometric sensor (e.g., a health care sensor) to collect biometric information (e.g., health data). For example, the electronic device may collect biometric information, such as blood pressure, a heartbeat, electrocardiogram, respiration, stress, or oxygen saturation using the biometric sensor.

The information described herein may be provided as the related art for the purpose of aiding the understanding of the disclosure. No assertion or determination is made with respect to the applicability of any of the above-mentioned contents as the prior art related to the disclosure.

### SUMMARY

In general, an electronic device may provide a blood pressure measurement based on a photoplethysmogram (PPG) signal obtained from a biometric sensor. In this regard, the electronic device may extract a feature point corresponding to a change in the body from the PPG signal and may use it for blood pressure measurement.

However, the PPG signal is vulnerable to a motion artifact. For example, in a situation where the PPG signal is obtained, a movement of a subject or a movement of the biometric sensor may degrade quality of the PPG signal. Such a PPG signal may degrade accuracy of blood pressure measurement.

Thus, at least one of various embodiments relates to a blood pressure measurement method for improving accuracy of measurement and an electronic device supporting the same.

Furthermore, at least one of various embodiments relates to a blood pressure measurement method for guiding an appropriate action capable of improving quality of a PPG signal, in a situation where the PPG signal with quality less than a certain level is obtained, and an electronic device supporting the same.

The technical problem to be solved in the disclosure is not limited to the above-mentioned technical problems, and other technical problems not described herein will be clearly understood by those skilled in the art from the description of the claims.

An electronic device according to various embodiments may include a first sensor which collects first information related to biometric information using a portion of a subject as a measurement area, a second sensor which collects second information related to a posture of the electronic device, a third sensor which collects third information related to a temperature of the measurement area, at least one processor, and a memory. The at least one instruction, when executed by the at least one processor, may cause the electronic device to measure signal quality of a biometric signal represented by the first information and, in response to determining the signal quality is below a specified level, obtain a first result indicating whether the posture of the electronic device corresponds to a designated reference posture based on the second information, obtain a second result indicating whether the temperature of the measurement area corresponds to a designated reference temperature based on the third information, and selectively provide a first guide related to the posture of the electronic device or a second guide related to the temperature of the measurement area, based on the first result and the second result.

An electronic device according to various embodiments may include a biometric sensor which collects biometric information using a portion of a subject as a measurement area, a temperature sensor which collects temperature information associated with the measurement area, at least one processor, and a memory. The memory may include at least one instruction. The at least one instruction, when executed by the at least one processor, may cause the electronic device to estimate a value of a first measurement parameter related to blood pressure measurement based on the biometric information, measure a first temperature of the measurement area based on the temperature information, obtain a second value of the first measurement parameter by correcting the value of the first measurement parameter based on the first temperature and a second temperature stored in the electronic device, and provide a blood pressure measurement based on the second value of the first measurement parameter.

An operating method of an electronic device according to various embodiments may include collecting first information related to biometric information using a portion of a subject as a measurement area, measuring signal quality of a biometric signal represented by the first information, and, in response to determining the signal quality is below a specified level, obtaining second information related to a posture of the electronic device and third information related to a temperature of the measurement area, obtaining a first result indicating whether the posture of the electronic device corresponds to a designated reference posture based on the second information, obtaining a second result indicating whether the temperature of the measurement area corresponds to a designated reference temperature based on the third information, and selectively providing a first guide related to the posture of the electronic device or a second guide related to the temperature of the measurement area, based on the first result and the second result.

A computer-readable storage medium according to various embodiments may store at least one instruction to collect first information related to biometric information using a portion of a subject as a measurement area, measure signal quality of a biometric signal represented by the first information, and, in response to determining the signal quality is below a specified level, obtain second information related to a posture of the electronic device and third information related to a temperature of the measurement area, obtain a first result indicating whether the posture of the electronic device corresponds to a designated reference posture based on the second information, obtain a second result indicating whether the temperature of the measurement area corresponds to a designated reference temperature based on the third information, and selectively provide a first guide related to the posture of the electronic device or a second guide related to the temperature of the measurement area, based on the first result and the second result.

The electronic device according to various embodiments disclosed in the disclosure may generate information (e.g., text information, image information, audio information) which guides a user in performing an appropriate action capable of improving quality of a PPG signal in a current measurement situation, when the PPG signal with quality less than a certain level is obtained, to use the PPG signal with the quality of the certain level for blood pressure measurement and as a result, may improve the accuracy of the blood pressure measurement.

The effects that are achieved through the disclosure may not be limited to the effects described herein, and other advantages not described herein may be more clearly understood from the following detailed description by those skilled in the art to which the disclosure pertains.

### BRIEF DESCRIPTION OF THE DRAWINGS

FIG. 1 is a block diagram of an electronic device in a network environment according to various embodiments;

FIGS. 2A and 2B are drawings illustrating the form of an electronic device according to various embodiments;

FIG. 3 is a drawing schematically illustrating a configuration of an electronic device according to various embodiments;

FIG. 4 is a drawing for describing a blood pressure measurement operation of an electronic device according to various embodiments;

FIG. 5 is a drawing for describing quality of a PPG signal according to a motion artifact;

FIG. 6 is a drawing for describing a guide for motion artifact reduction according to various embodiments;

FIG. 7 is a drawing for describing a PPG signal, quality of which is degraded by various factors other than a motion artifact;

FIGS. 8 and 9 are drawings for describing a guide for improving quality of a PPG signal according to various embodiments;

FIGS. 10A and 10B are drawings for describing a blood pressure measurement operation of an electronic device according to various embodiments;

FIG. 11 is a flowchart illustrating an operation of an electronic device according to various embodiments;

FIG. 12 is a flowchart illustrating an operation of providing a guide in an electronic device according to various embodiments;

FIG. 13 is a flowchart illustrating an operation of an electronic device according to various embodiments; and

FIG. 14 is a flowchart illustrating an operation of an electronic device according to various embodiments.

### DETAILED DESCRIPTION

Hereinafter, various embodiments of the disclosure will be described with reference to the accompanying drawings. However, this is not intended to limit the technology described in the disclosure to specific embodiments, and should be understood to include various modifications, equivalents, and/or alternatives to the embodiments of the disclosure. In relation to the description of the drawings, similar reference numbers may be used for similar components.

Terms such as, for example, first, second, and the like may be used to describe various components, but the components should not be limited by the terms. The terms as used herein may distinguish one component from other components and are not to be limited by the terms. For example, without departing from the scope of the present disclosure, a first component may be referred to as a second component, and similarly, the second component may also be referred to as the first component. The terms of a singular form may include plural forms unless otherwise specified.

The terminology used herein is for the purpose of describing particular embodiments and is not intended to be limiting. As used herein, “a,” “an,” “the,” and “at least one” do not denote a limitation of quantity, and are intended to include both the singular and plural, unless the context clearly indicates otherwise. For example, “an element” has the same meaning as “at least one element,” unless the context clearly indicates otherwise. “At least one” is not to be construed as limiting “a” or “an.” “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, or components.

The term “adjacent” is used to describe the relative positions of various components. The term may refer to a directly adjacent relationship between components but is not necessarily limited or intended to mean that the components are directly adjacent each other. The term may mean that the components are situated in respective regions that are directly adjacent and the components may therefore be merely mutually proximal and be separated by a gap. For example, the term “adjacent” or “adjacent to,” as used herein, may include “next to,” “adjoining,” “in contact with,” or “in proximity to.”

Spatially relative terms, such as “beneath,” “below,” “lower,” “above,” “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations) and the spatially relative descriptors used herein interpreted accordingly.

Embodiments are described herein with reference to cross section illustrations that are schematic illustrations of example embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

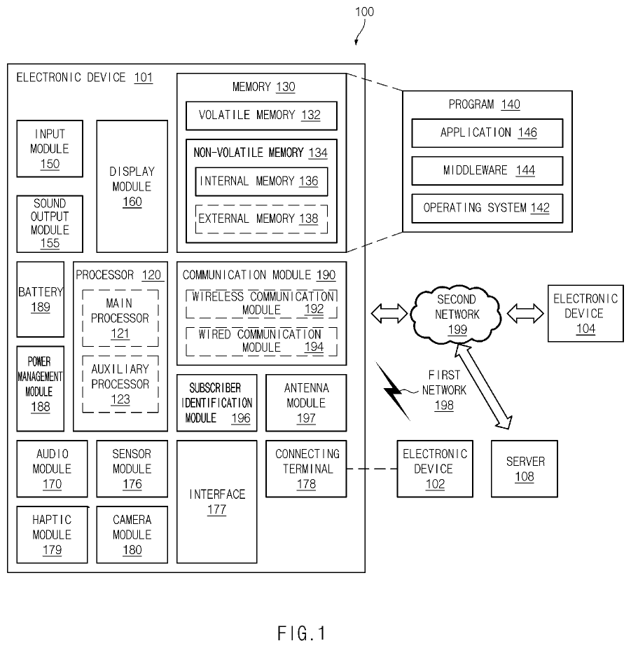

FIG. 1 is a block diagram illustrating an electronic device 101 in a network environment 100 according to various embodiments.

Referring to FIG. 1, the electronic device 101 in the network environment 100 may communicate with an electronic device 102 via a first network 198 (e.g., a short-range wireless communication network), or at least one of an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one of the components (e.g., the connecting terminal 178) may be omitted from the electronic device 101, or one or more other components may be added in the electronic device 101. In some embodiments, some of the components (e.g., the sensor module 176, the camera module 180, or the antenna module 197) may be implemented as a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. In an example in which the electronic device 101 includes the main processor 121 and the auxiliary processor 123, the auxiliary processor 123 may be adapted to consume less power than the main processor 121, or to be specific to a specified function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. An artificial intelligence model may be generated by machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, a key (e.g., a button), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display module 160 may include, for example, a display, a hologram device, or a projector and control circuitry to control a corresponding one of the display, hologram device, and projector. According to an embodiment, the display module 160 may include a touch sensor adapted to detect a touch, or a pressure sensor adapted to measure the intensity of force incurred by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or a movement) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device via the first network 198 (e.g., a short-range communication network, such as Bluetooth™, wireless-fidelity (Wi-Fi) direct, or infrared data association (IrDA)) or the second network 199 (e.g., a long-range communication network, such as a legacy cellular network, a 5G network, a next-generation communication network, the Internet, or a computer network (e.g., LAN or wide area network (WAN)). These various types of communication modules may be implemented as a single component (e.g., a single chip), or may be implemented as multi components (e.g., multi chips) separate from each other. The wireless communication module 192 may identify and authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4G network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device) of the electronic device 101. According to an embodiment, the antenna module 197 may include an antenna including a radiating element composed of a conductive material or a conductive pattern formed in or on a substrate (e.g., a printed circuit board (PCB)). According to an embodiment, the antenna module 197 may include a plurality of antennas (e.g., array antennas). In such a case, at least one antenna appropriate for a communication scheme used in the communication network, such as the first network 198 or the second network 199, may be selected, for example, by the communication module 190 (e.g., the wireless communication module 192) from the plurality of antennas. The signal or the power may then be transmitted or received between the communication module 190 and the external electronic device via the selected at least one antenna. According to an embodiment, another component (e.g., a radio frequency integrated circuit (RFIC)) other than the radiating element may be additionally formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. Each of the electronic devices 102 or 104 may be a device of a same type as, or a different type, from the electronic device 101. According to an embodiment, all or some of operations to be executed at the electronic device 101 may be executed at one or more of the electronic device 102, the electronic device 104, or the server 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or healthcare) based on 5G communication technology or IoT-related technology.

According to an embodiment, the processor 120 (e.g., processing circuit) may be implemented as one or more integrated circuit (or circuitry) chips and may perform various data processing operations. The processor 120 may include at least one electrical circuit and may individually or collectively distribute and process instructions (or programs, data) stored in the memory 130. The processor 120 may include a processor assembly including one or more processing circuits. The processor 120 may include any processing circuit operative to control the performance and operations of one or more components of the electronic device 101 (e.g., the memory 130, the display module 160, the sensor module 176 (e.g., a sensor), the camera module 180 (e.g., an image sensor), and/or the communication module 190 (e.g., a communication circuit)). For example, the processor 120 (e.g., an application processor (AP)) may be implemented as a system on chip (SoC) (e.g., a single chip or a chipset). For example, the processor 120 may be implemented as a plurality of cores (or at least one core circuit), a plurality of chips, or a plurality of chipsets. For example, the processor 120 may include one or more processing circuits. For example, the processor 120 may include one or more processing circuits configured to individually and/or collectively perform various functions of the disclosure. As a non-limiting example, at least part of the processor 120 may be included in a first chip of the electronic device 101, and at least another part of the processor 120 may be included in a second chip of the electronic device 101 different from the first chip of the electronic device 101.

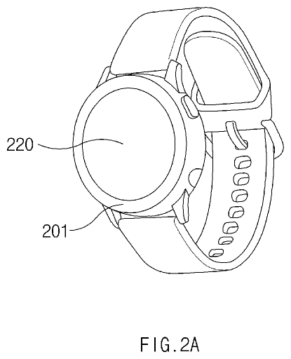

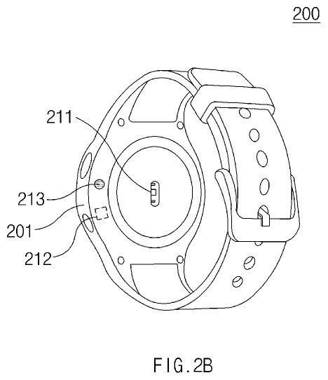

FIGS. 2A and 2B are drawings illustrating the form of an electronic device 200 according to various embodiments.

Referring to FIGS. 2A and 2B, the electronic device 200 (e.g., an electronic device 101 of FIG. 1) may be a wearable device in the form of a watch worn on a body part (e.g., a wrist). However, this is illustrative and various embodiments are not limited thereto. For example, the electronic device 200 may be implemented as wearable devices in various forms, such as a band or ring form.

According to an embodiment, the electronic device 200 may be implemented in various forms. According to an embodiment, as illustrated, the electronic device 200 may be implemented in a circular shape attachable to a part of the body.

However, this is illustrative and various embodiments are not limited thereto. For example, the electronic device 200 may be implemented in various forms, such as a rectangle, a square, and an oval, which are attachable to a body part of a user. Furthermore, the electronic device 200 may be implemented in the form of having a curved surface for holding by the user.

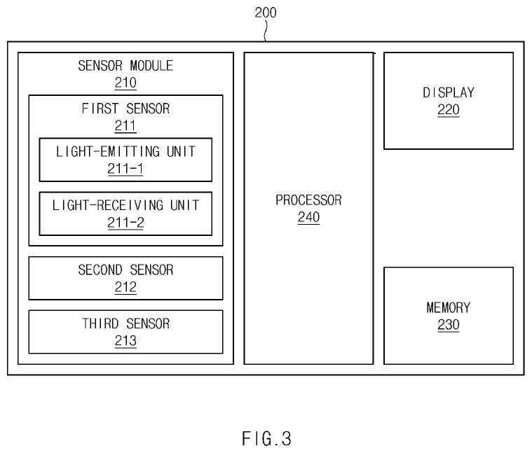

According to various embodiments, the electronic device 200 may include various components. The various components may be at least some of the components of an electronic device 101 illustrated in FIG. 1. In this regard, a description will be given in detail with reference to FIG. 3 below.

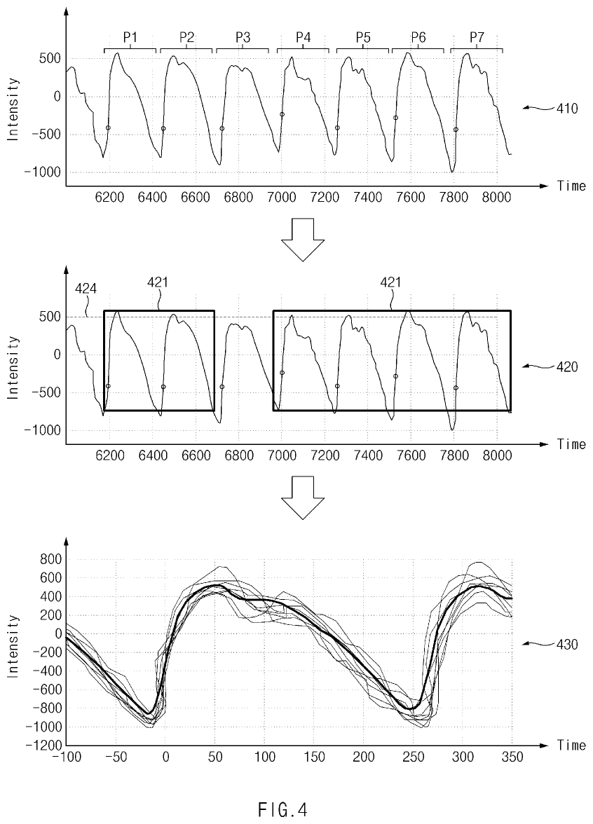

FIG. 3 is a drawing schematically illustrating a configuration of an electronic device 200 according to various embodiments. Next, FIG. 4 is a drawing for describing a blood pressure measurement operation of an electronic device 200 according to various embodiments.

Referring to FIGS. 2A to 3, the electronic device 200 according to various embodiments may be composed of a housing 201 (e.g., at least one housing 201), a sensor module 210 (e.g., at least one sensor module 210), a display 220 (e.g., at least one display 220), a memory 230 (e.g., at least one memory 230), and a processor 240 (e.g., at least one processor 240).

The above-mentioned components of the electronic device 200 are one embodiment and various embodiments are not limited thereto. For example, the electronic device 200 may be implemented as having more components than the components illustrated in FIG. 3 or having less components than them. For example, at least some (e.g., an input module 150, a battery 189, or an antenna module 197) of the components of the electronic device 101 illustrated in FIG. 1 may be included in the configuration of the electronic device 200. Furthermore, at least one of the components of the electronic device 200, which are illustrated in FIG. 3, may be integrated with another component.

According to various embodiments, the housing 201 may form the appearance of the electronic device 200. For example, the housing 201 may include a first surface (e.g., a front surface), a second surface (e.g., a rear surface), and a third surface (e.g., a side surface) surrounding a space between the first surface and the second surface.

Such a housing 201 may provide a space for mounting the components (e.g., the sensor module 210, the display 220, the memory 230, and the processor 240) of the electronic device 200. According to an embodiment, some (e.g., a part of the sensor module 210 and the display 220) of the components of the electronic device 200, which are mounted into the housing 201, may be exposed via at least a part of the housing 201.

According to various embodiments, the sensor module 210 may be composed of a plurality of sensors. According to an embodiment, the sensor module 210 may include a first sensor 211 (e.g., at least one first sensor 211) configured to obtain biometric information, a second sensor 212 (e.g., at least one second sensor 212) configured to obtain information related to a posture and/or a wearing state of the electronic device 200, and a third sensor 213 (e.g., at least one third sensor 213) configured to obtain temperature information about a specific part (e.g., a measured part) of the body. For example, the sensor module 210 may be the sensor module 176 illustrated in FIG. 1.

The “posture” of the electronic device 200 as described herein may refer to the position and orientation of the electronic device 200, for example, in one or more dimensions (e.g., x-axis, y-axis, z-axis).

The above-mentioned configuration of the sensor module 210 is one embodiment and various embodiments are not limited thereto. For example, the sensor module 210 may be implemented with more sensors than the above-mentioned sensors. For example, at least one electrode connected with the first sensor 211 and configured to obtain biometric information about a part of the body may be added as the configuration of the sensor module 210. Furthermore, the sensor module 210 may be implemented with less sensors than the above-mentioned sensors. According to an embodiment, at least one of the first to third sensors 211 to 213 may be implemented with a plurality of sensors.

According to various embodiments, the first sensor 211 may obtain a pulse wave signal from a measurement area (a measuring part) (e.g., a wrist). The pulse wave signal may be a PPG signal. According to an embodiment, the first sensor 211 may be composed of a light-emitting unit 211-1 and a light-receiving unit 211-2.

According to an embodiment, the light-emitting unit 211-1 may be composed of a first light-emitting element for radiating light of a first wavelength (e.g., a red color (wavelength: 600 nm~700 nm)) to the measurement area and a second light-emitting element for radiating light of a second wavelength (e.g., an infrared ray (wavelength: 780 nm~1000 μm)) to the measurement area. For example, the first light-emitting element and the second light-emitting element constituting the light-emitting unit 211-1 may be implemented using a light emitting diode (LED), an organic light emitting diode (OLED), quantum dot light-emitting diodes (QLED), a laser diode, or a fluorescent body.

However, this is illustrative and the disclosure is not limited thereto. For example, the light-emitting unit 211-1 may further include one or more additional light-emitting element of a wavelength (e.g., a blue wavelength, a green wavelength, or the like) which is the same as or different from the wavelengths of the first light-emitting element and the second light-emitting element.

According to an embodiment, the light-receiving unit 211-2 may receive light and may perform photoelectric conversion of the received light to generate a current signal. For example, the light-receiving unit 211-2 may include at least one light-receiving element for detecting the light of the first wavelength, which is radiated from the first light-emitting element, and the light of the second wavelength, which is radiated from the second light-emitting element. For example, the at least one light-receiving element may include a photo detector or a photo diode.

According to an embodiment, the light-receiving unit 211-2 may detect light which is radiated from the light-emitting unit 211-1 (e.g., the first light-emitting element and the second light-emitting element) and is reflected from the measurement area. In this regard, the light-receiving unit 211-2 and the light-emitting unit 211-1 may be arranged on the same surface.

According to an embodiment, the light-receiving unit 211-2 may detect light which is radiated from the light-emitting unit 211-1 and penetrates the measurement area. In this regard, the light-receiving unit 211-2 and the light-emitting unit 211-1 may be arranged to face each other.

However, this is illustrative and the disclosure is not limited thereto. The light-receiving unit 211-2 and the light-emitting unit 211-1 may be arranged in various forms. Furthermore, at least a part of the first sensor 211 may be exposed via a portion of the second surface (e.g., the rear surface) of the housing 201.

According to various embodiments, the second sensor 212 may obtain information related to an inertia force as at least a portion of information related to a posture (and/or a movement) of the electronic device 200. For example, the second sensor 212 may be composed of at least one of an acceleration sensor, a gyro sensor, a gesture sensor, or a geomagnetic sensor. According to various embodiments, the second sensor 212 may obtain information related to proximity to the measurement area as at least a portion of information related to the wearing state of the electronic device 200. For example, the second sensor 212 may further include a proximity sensor.

According to various embodiments, the third sensor 213 may obtain temperature information about the measurement area. The measurement area may be a part (e.g., a wrist) of the body adhering to (e.g., in contact with) the first sensor 211 in the state in which the electronic device 200 is worn on the body. For example, the third sensor 213 may include a temperature sensor configured to facilitate temperature measurement for a specific part (e.g., the measurement area) of the body, rather than a body temperature.

According to an embodiment, the third sensor 213 may be disposed on the same surface as the first sensor 211, toward the measurement area. For example, the third sensor 213 may be disposed adjacent to the first sensor 211. For example, the third sensor 213 may be exposed via a portion of the second surface (e.g., the rear surface) of the housing 201. For example, the third sensor 213 may be configured to obtain temperature information about the measurement area in a contact manner or may obtain the temperature information about the measurement area in a non-contact manner.

According to various embodiments, the display 220 may be used to provide information processed by and generated by the electronic device 200. According to an embodiment, the display 220 may display a screen or a user interface for the information processed by and generated by the electronic device 200. For example, the display 220 may be exposed via a portion of the first surface (e.g., the front surface) of the housing 201. For example, the display 220 may be the display module 160 illustrated in FIG. 1.

According to various embodiments, the memory 230 may store various pieces of data used by components of the electronic device 200. According to an embodiment, instructions to perform a function (e.g., an operation) of the electronic device 200 may be stored in the memory 230. For example, the memory 230 may be the memory 130 illustrated in FIG. 1.

According to various embodiments, the processor 240 may be operatively connected with the sensor module 210, the display 220, and the memory 230 and may control various components (e.g., hardware or software components) of the electronic device 200. For example, the processor 240 may include circuitry, such as a central processing unit (CPU), a microprocessor unit (MPU), an application processor (AP), a communication processor (CP), a system on chip (SoC), or an integrated circuit (IC). For example, the processor 240 may be the processor 120 illustrated in FIG. 1.

According to various embodiments, the processor 240 may support a function related to blood pressure measurement of the electronic device 200.

According to an embodiment, the processor 240 may provide a blood pressure measurement (i.e., measure blood pressure) based on various pieces of information obtained via the sensor module 210 (e.g., the first sensor 211). For example, the processor 240 may provide a blood pressure measurement based on the PPG signal obtained via the first sensor 211.

In this regard, referring to FIG. 4, the processor 240 may obtain (410) a plurality of PPG signals (e.g., waveforms of the PPG signal) P1 to P7, noise of which is removed by the electronic device 200 by amplifying or filtering the signal of the first sensor 211, and may extract (420) a plurality of PPG signals 421 meeting a designated condition among the plurality of obtained PPG signals P1 to P7 to use them for blood pressure measurement.

According to an embodiment, the plurality of PPG signals 421 meeting the designated condition may be signals with a peak of a threshold 424 or more. The processor 240 may overlap the plurality of extracted PPG signals 421 with one another and may use a feature point extracted from the overlapping PPG signals 430 for blood pressure measurement. For example, the processor 240 may provide a blood pressure measurement based on the result of comparing the feature point extracted from the overlapped PPG signal with a stored reference feature point.

For example, the PPG signal may include or be formed from a progression wave and a reflected wave which overlap each other. The progression wave occurs and progresses from a heart, and the reflected wave returns from a terminal part. Such a PPG signal in which the progression wave and the reflected wave overlap each other may reflect a state, a blood pressure, or the like of the cardiovascular system. In this regard, the processor 240 may apply an ensemble average to the PPG signal to extract various feature points and may use the result for blood pressure measurement. For example, the processor 240 may estimate a value of a measurement parameter related to blood pressure measurement, such as a cardiac output and/or total peripheral resistance (TPR), based on the various feature points and may measure at least one of a systolic blood pressure or a diastolic blood pressure based on the estimated value.

However, the above-mentioned blood pressure measurement method is illustrative, and embodiments of the present disclosure are not limited thereto. In conjunction with blood pressure measurement, various well-known technologies may be considered as various embodiments described in the disclosure.

As described herein, the processor 240 according to various embodiments may obtain a PPG signal as a part of the blood pressure measurement operation. However, a motion artifact associated with a movement (e.g., a movement of the subject or a movement of the first sensor 211) which occurs in the situation where the PPG signal is obtained may cause deterioration in quality of the PPG signal.

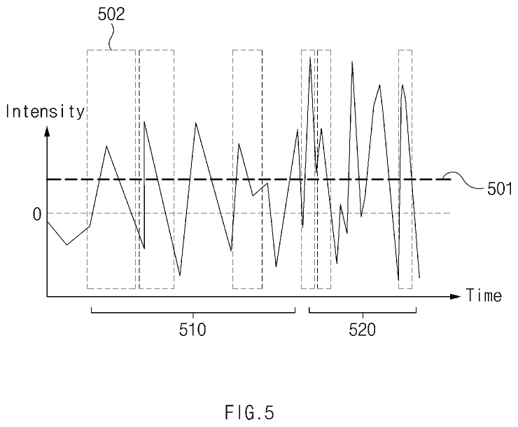

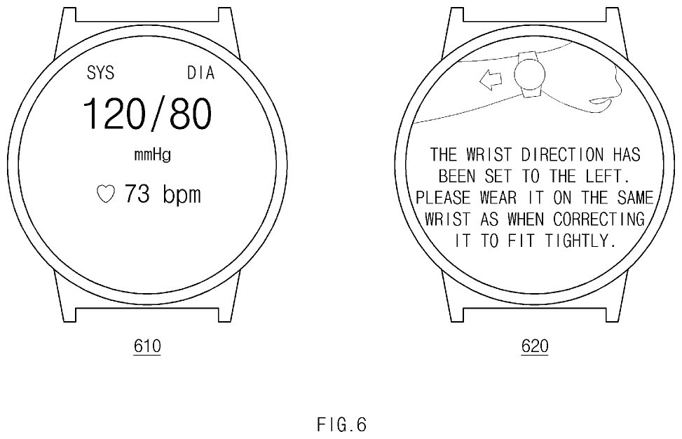

According to various embodiments, the processor 240 may generate information (e.g., text information, image information, audio information) which guides a user in performing an appropriate action capable of removing the motion artifact to improve reliability of blood pressure measurement. In this regard, a description will be given in detail with reference to FIGS. 5 and 6 below.

FIG. 5 is a drawing for describing quality of a PPG signal according to a motion artifact. Next, FIG. 6 is a drawing for describing a guide for motion artifact reduction according to various embodiments.

Referring to FIG. 5, when a motion artifact does not occur in a situation where a PPG signal is obtained (e.g., when a subject is in a posture suitable for measurement or an electronic device 200 is normally worn according to a position or orientation which supports the measurement), as illustrated in 510 of FIG. 5, the electronic device 200 may obtain a PPG signal with quality of a certain level. Based on the PPG signal with quality of the certain level, the electronic device 200 can extract a meaningful feature point relatively easily and/or estimate a value of a measurement parameter relatively easily, compared to a case in which the quality of the PPG signal is less than the certain level. For example, in FIG. 5, for the case where a motion artifact does not occur, the electronic device 200 may obtain a PPG signal with a peak intensity greater than a threshold intensity value 501 or more and having periodic signals 502 of a similarity greater than or equal to the certain level with respect to one another.

However, when the motion artifact occurs in the situation where the PPG signal is obtained (e.g., when the subject does not assume or maintain the posture suitable for the measurement or the electronic device 200 is abnormally worn according to a position or orientation which does not support the measurement), as illustrated in 520 of FIG. 5, a PPG signal, quality of which is degraded by the motion artifact (e.g., in which the electronic device 200 is unable to extract the meaningful feature (and/or the electronic device 200 is unable to estimate the value of the measurement parameter)), may be obtained. For example, for the case where a motion artifact does occur, the electronic device 200 may obtain a PPG signal with a peak intensity greater than the threshold intensity value 501 or more and a having periodic signals 502 of a similarity less than the certain level with respect to one another.

In this regard, a processor 240 according to various embodiments may identify a situation where the motion artifact occurs based on various pieces of information obtained via a sensor module 210 (e.g., a second sensor 212). For example, the processor 240 may determine a situation where the movement of the electronic device 200 is greater than or equal to the certain level or the movement of a designated pattern is detected as the situation where the motion artifact occurs. For example, the processor 240 may compare information obtained from the second sensor 212 with stored reference information to identify the situation where the motion artifact occurs.

According to an embodiment, in a situation where the motion artifact does not occur, the processor 240 may provide a blood pressure measurement which has reliability satisfying a reliability threshold and may provide the blood pressure measurement as a measurement result. For example, as in 610 of FIG. 6, the processor 240 may provide a systolic blood pressure (e.g., 120 mmHg) and a diastolic blood pressure (e.g., 80 mmHg) as visual information. However, this is illustrative and various embodiments are not limited thereto. For example, the measurement result may be provided in various forms, such as audible information and tactile information.

According to an embodiment, in the situation where the motion artifact occurs, the processor 240 may generate information (e.g., text information, image information, audio information) which guides a user in performing an appropriate action capable of removing the motion artifact from the PPG signal in the current measurement situation. According to an embodiment, the processor 240 may generate and provide at least one of text information and image information as a guide (i.e., instruction) for inducing a user to assume a posture which matches a predetermined blood pressure measurement posture and/or inducing the user to position a measurement device (e.g., the electronic device 200, a first sensor 211 of the electronic device 200) to a predetermined measurement position.

A “guide” as provided by the processor 240 may include an indicator, text, an image, audio, haptic feedback, or the like which includes instructions for a user with respect to obtaining a blood pressure measurement described herein.

For example, as in 620 of FIG. 6, the processor 240 may provide a guide for inducing the electronic device 200 to be normally worn (e.g., the electronic device 200 may display a message ‘Please wear the electronic device on your wrist.’). Additionally or alternatively, the processor 240 may provide a guide for inducing a posture suitable for obtaining the measurement (e.g., Please do not move during the measurement.)

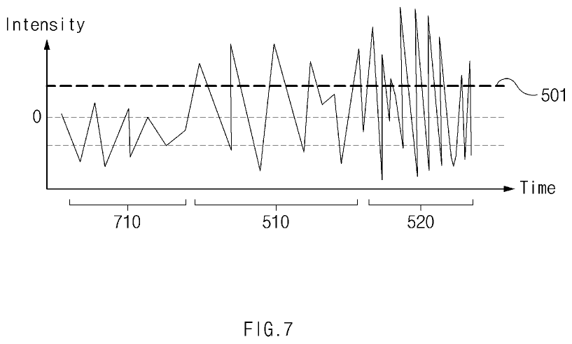

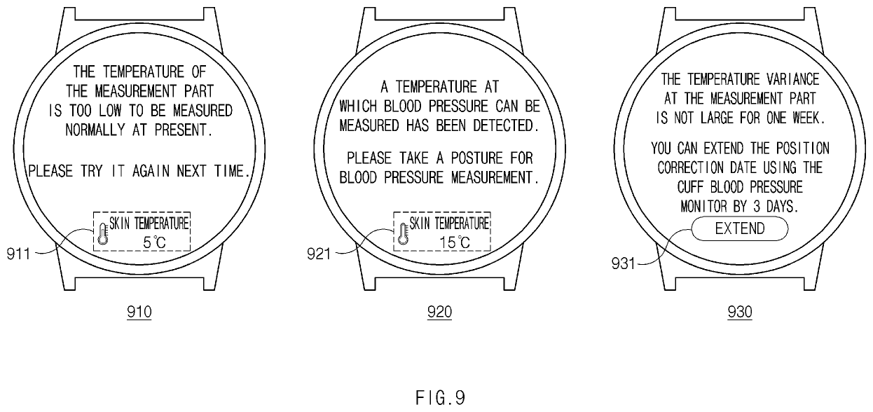

As described herein, the processor 240 may generate information (e.g., text information, image information, audio information) which guides a user in performing an appropriate action capable of removing the motion artifact to improve reliability of blood pressure measurement. However, deterioration in quality of the PPG signal may be caused by various factors other than the motion artifact. Thus, the processor 240 according to various embodiments may identify a factor which degrades the quality of the PPG signal and may generate information (e.g., text information, image information, audio information) which guides a user in performing an appropriate action corresponding to the identified result. In this regard, a description will be given in detail with reference to FIGS. 7 and 9 below.

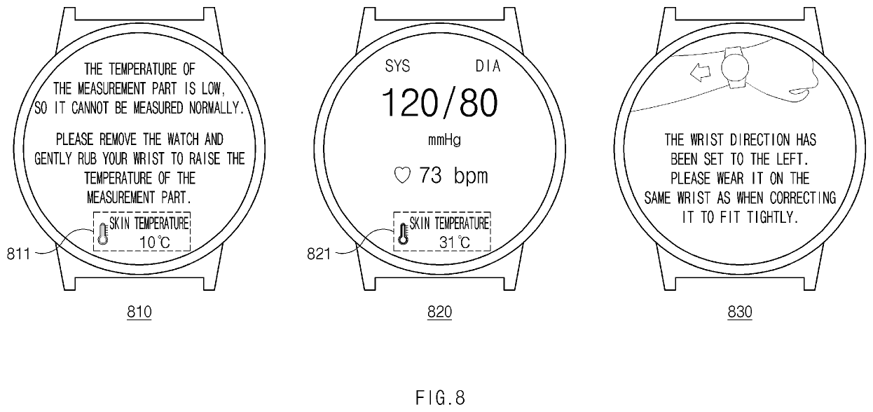

FIG. 7 is a drawing for describing a PPG signal, quality of which is degraded by various factors other than a motion artifact. Next, FIGS. 8 and 9 are drawings for describing a guide for improving quality of a PPG signal according to various embodiments.

As described herein, the PPG signal may include or be formed from a progression wave (which occurs and progresses from a heart) and a reflected wave (which returns from a terminal part) which overlap each other. Furthermore, the blood vessel of a person constricts and expands. For example, the blood vessel constricts to minimize heat loss via the blood vessel in a cold environment. This may act as a factor which interferes with obtaining a PPG signal with good quality.

In an example in which the temperature of the measurement area decreases to less than a certain temperature in the situation where the PPG signal is obtained, although the motion artifact does not occur (e.g., when a subject takes a posture suitable for measurement or an electronic device 200 is normally worn), the quality of the PPG signal may be degraded (e.g., the electronic device 200 is unable to extract a meaningful feature point from the PPG signal). For example, as illustrated in 710 of FIG. 7, a PPG signal with a similarity between periodic signals 502, which is greater than or equal to a certain level, or a peak less than a threshold intensity value 501 may be obtained.

According to various embodiments, a processor 240 may identify a factor which degrades quality of the PPG signal based on various pieces of information obtained via a sensor module 210 (e.g., a third sensor 213) and may generate information (e.g., text information, image information, audio information) which guides a user in performing an appropriate action corresponding to the identified result. For example, the processor 240 may identify a temperature of the measurement area, which is less than a certain temperature, as the factor which degrades the quality of the PPG signal.

According to an embodiment, when detecting the temperature of the measurement area, which is less than the certain temperature, as in 810 of FIG. 8, the processor 240 may generate information (e.g., text information, image information, audio information) which guides a user in performing an appropriate action for increasing the temperature of the measurement area (e.g., Please rub the measurement area to raise the temperature.). Additionally or alternatively, the processor 240 may output information 811 related to the measured temperature of the measurement area (e.g., a current temperature of the measurement area or a temperature which should increase to obtain a PPG signal with good quality) together with the guide.

According to an embodiment, when detecting a temperature of the measurement area, which is greater than or equal to the certain temperature, the processor 240 may determine whether a motion artifact occurs in the situation where the PPG signal is obtained and may provide a guide corresponding to the determined result. For example, the processor 240 may obtain information related to a posture and/or a wearing state of the electronic device 200 via the sensor module 210 (e.g., a second sensor 212) in the situation where the PPG signal is obtained and may determine whether the motion artifact occurs based on it.

According to an embodiment, when the motion artifact does not occur in the situation where the PPG signal is obtained, the processor 240 may provide a blood pressure measurement which has a reliability satisfying a reliability threshold. Thus, the processor 240 may provide the blood pressure measurement as a measurement result. For example, as in 820 of FIG. 8, the processor 240 may provide a systolic blood pressure (e.g., 120 mmHg) and a diastolic blood pressure (e.g., 80 mmHg) as visual information. Additionally or alternatively, the processor 240 may provide information 821 related to the current temperature of the measurement area together with the measurement result.

According to an embodiment, when the motion artifact occurs in the situation where the PPG signal is obtained, the processor 240 may be unable to provide a blood pressure measurement which has a reliability satisfying a reliability threshold. Thus, the processor 240 may generate information (e.g., text information, image information, audio information) which guides a user in performing an appropriate action capable of removing the motion artifact from the PPG signal in the current measurement situation. For example, as in 830 of FIG. 8, the processor 240 may provide a guide for inducing the electronic device 200 to be normally worn (e.g., Please wear the electronic device on your wrist.). Additionally or alternatively, the processor 240 may provide information related to the current temperature of the measurement area together with the guide.

Additionally or alternatively, the processor 240 according to various embodiments may provide various guides based on the temperature of the measurement area.

According to an embodiment, although a user takes a guide action (e.g., an action which rubs the measurement area), a blood vessel which constricts to a certain level or more due to a temperature of a surrounding environment may fail to easily expand. In this case, it may be difficult to normally provide a blood pressure measurement.

In this regard, when the temperature of the measurement area is less than a limit temperature, the processor 240 according to various embodiments may guide a situation where the electronic device 200 is unable to normally measure the blood pressure. For example, as illustrated in 910 of FIG. 9, for the situation where it is difficult to normally measure the blood pressure in the current state, the processor 240 may generate information (e.g., text information, image information, audio information) which guides a user in performing an appropriate action (e.g., Please try the measurement again next time.). According to an embodiment, the processor 240 may output information 911 related to the measured temperature of the measurement area together with the guide.

Additionally or alternatively, the processor 240 according to various embodiments may provide various guides (e.g., instructional text information, image information, audio information, or the like for performing an action) based on a change in the temperature of the measurement area.

According to an embodiment, the blood vessel which constricts to the certain level or more due to the temperature of the surrounding environment may expand to the certain level due to the guided action. In other words, the temperature of the measurement area, which is less than or equal to the certain level, may increase to the certain level. In this regard, when the change in the temperature of the measurement area, which meets a designated condition, is detected, the processor 240 may guide a situation where it is possible to normally measure the blood pressure. For example, as illustrated in 920 of FIG. 9, the processor 240 may generate a guide indicating the situation where it is possible to normally measure the blood pressure in the current state has been detected (e.g., A temperature at which blood pressure can be measured has been detected.). The guide may further include additional instructions for the user (e.g., Please take a posture for blood pressure measurement). According to an embodiment, the processor 240 may output information 921 related to the measured temperature of the measurement area together with the guide.

Additionally or alternatively, the processor 240 according to various embodiments may generate information (e.g., text information, image information, audio information) which guides a user in performing an appropriate action for maintaining or adjusting a blood pressure correction time point, based on the temperature of the measurement area.

According to an embodiment, in measuring a blood pressure, the processor 240 may use a scheme for indirectly estimating the blood pressure rather than a scheme for actually and directly measuring the blood pressure of a coronary artery. Thus, to measure an accurate blood pressure, the processor 240 is capable of performing an operation (e.g., calibration) for correcting the blood pressure estimated from the PPG signal to a real blood pressure (e.g., a blood pressure measured by a cuff blood pressure monitor) according to a designated period (e.g., at intervals of one month).

In this regard, the processor 240 according to various embodiments may provide a guide for meeting or adjusting a blood pressure correction time point based on body change information of the measurement area.

According to an embodiment, when the change in the temperature of the measurement area is greater than or equal to the certain level during a designated duration (e.g., 20 days), the processor 240 may perform a correction operation at a designated correction period.

According to an embodiment, when the change in the temperature of the measurement area (temperature variance in the measurement area) is less than the certain level during the designated duration (e.g., 7 days, 20 days), the processor 240 may generate information (e.g., text information, image information, audio information) which informs a user that it is possible to adjust the blood pressure correction time point, and the information may guide the user in performing an appropriate action which adjusts the blood pressure correction time point. For example, as illustrated in 930 of FIG. 9, because the change in the temperature of the measurement area is not large (e.g., less than a threshold temperature value), the electronic device 200 may generate information for guiding the user in the situation where it is possible to extend the blood pressure correction time point. In this regard, the processor 240 may provide a menu 931 configured to generate an instruction to extend blood pressure correction and may adjust the blood pressure correction time point based on a correction period, when an input to it is detected.

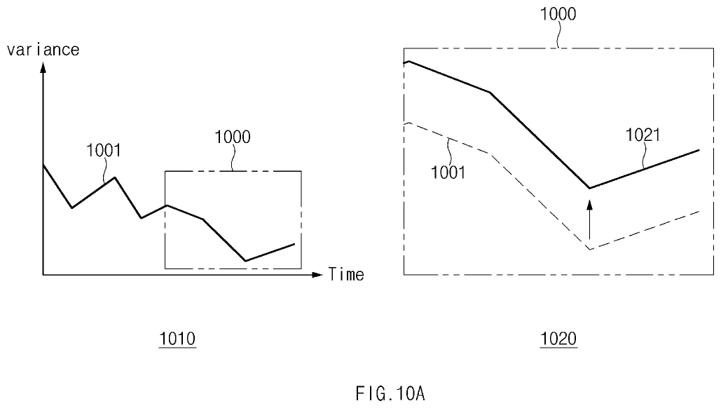

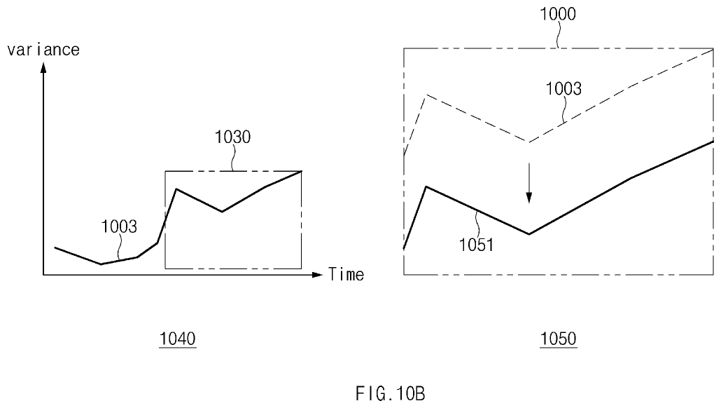

As described herein, the processor 240 may improve reliability of blood pressure measurement by providing the guide. The processor 240 according to various embodiments may correct a measurement parameter (e.g., total peripheral resistance (TPR) and/or a cardiac output) used for blood pressure measurement based on the temperature of the measurement area to improve reliability of the blood pressure measurement. In this regard, a description will be given in detail with reference to FIGS. 10A and 10B below.

FIGS. 10A and 10B are drawings for describing a blood pressure measurement operation of an electronic device 200 according to various embodiments.

As described herein, in measuring a blood pressure, a processor 240 according to various embodiments may estimate a value of a measurement parameter (e.g., total peripheral resistance (TPR) (and/or a cardiac output)) related to blood pressure measurement based on a feature point extracted from a PPG signal and may measure at least one of a systolic blood pressure or a diastolic blood pressure based on it.

Furthermore, a blood vessel may constrict according to a change in temperature of a surrounding environment. Due to this, total peripheral resistance which is a measurement parameter measured in the measurement area may also change. For example, the total peripheral resistance measured in the measurement area increases as the blood vessel constricts and the total peripheral resistance measured in the measurement area decreases as the blood vessel expands. As such, the total peripheral resistance changed by the blood vessel constriction and expansion may degrade reliability of blood pressure measurement. The total peripheral resistance changed according to the above-mentioned blood vessel constriction and total peripheral resistance corrected based on a temperature variance of the measurement area, which will be described herein, are one example of the measurement parameter and various embodiments are not limited thereto.

In this regard, the processor 240 according to various embodiments may correct total peripheral resistance measured in the measurement area based on the temperature variance of the measurement area.

The processor 240 according to various embodiments may determine whether to correct total peripheral resistance based on the result of comparing a first temperature of the measurement area, which is stored at the blood pressure correction time point, with a second temperature of the measurement area, which is measured when obtaining the PPG signal.

According to an embodiment, as illustrated in 1010 of FIG. 10A, first total peripheral resistance 1001 may decrease (1000) to a certain level or less by the constricted blood vessel. In this regard, as illustrated in 1020 of FIG. 10A, the processor 240 may generate second measured total peripheral resistance 1021 in which at least a portion of the first total peripheral resistance 1001 is increased to a certain level.

According to an embodiment, as illustrated in 1040 of FIG. 10B, third total peripheral resistance 1003 may increase (1030) to the certain level or more by the expanded blood vessel. In this regard, as illustrated in 1050 of FIG. 10B, the processor 240 may generate fourth measured total peripheral resistance 1051 in which the third total peripheral resistance 1003 decreases to the certain level.

As described herein, the processor 240 according to various embodiments may provide a blood pressure measurement based on the corrected total peripheral resistance to obtain a measurement result with higher reliability.

As described herein, the electronic device 200 according to various embodiments may include the one processor 240. In this case, the processor 240 may execute instructions stored in the memory 230 to perform a function (e.g., an operation) of the electronic device 200.

However, this is illustrative and various embodiments are not limited thereto. For example, the electronic device 200 according to various embodiments may include the plurality of processors 240. In this case, some of the plurality of processors 240 may execute the instructions stored in the memory 230 to perform some functions of the electronic device 200 and the others of the plurality of processors 240 may execute the instructions stored in the memory 230 to perform the other functions of the electronic device 200.

According to an embodiment, the electronic device 200 according to various embodiments may include the plurality of memories 230. In this case, instructions to perform some functions of the electronic device 200 may be stored in some of the plurality of memories 230 and instructions to perform the other functions of the electronic device 200 may be stored in the others of the plurality of memories 230.

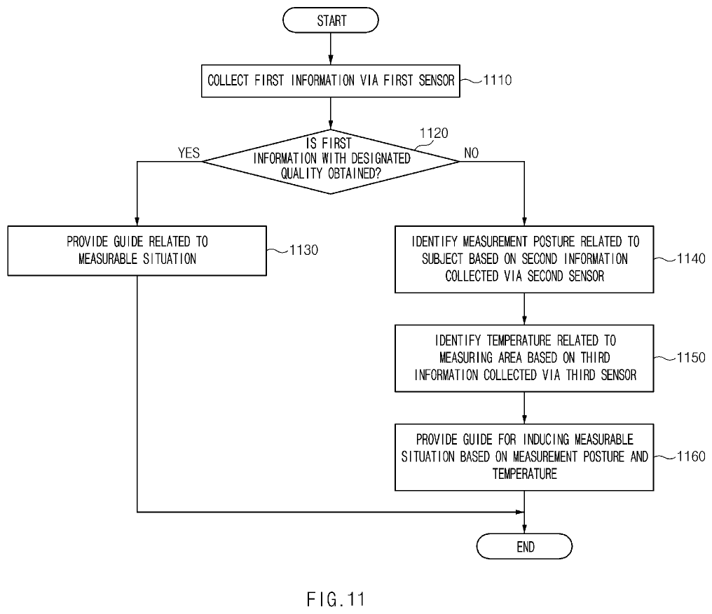

FIG. 11 is a flowchart illustrating an operation of an electronic device 200 according to various embodiments. Next, the respective operations in an embodiment below may be sequentially performed, but are not limited to being sequentially performed. For example, an order of the respective operations may be changed and at least two operations may be performed in parallel. Furthermore, at least one of the above-mentioned operations may be omitted according to an embodiment.

Referring to FIG. 11, in operation 1110, the electronic device 200 (e.g., a processor 240) according to various embodiments may collect (e.g., obtain) first information via a first sensor 211. According to an embodiment, the electronic device 200 may obtain the first information represented by a biometric signal (e.g., a PPG signal) for a measurement area (e.g., a wrist).

According to various embodiments, in operation 1120, the electronic device 200 (e.g., the processor 240) may determine whether first information with designated quality is obtained. For example, the electronic device 200 may determine whether the biometric signal represented by the first information has a designated signal quality. As described with reference to 410 of FIG. 4, the first information with the designated quality (e.g., the biometric signal having the designated signal quality) may be some of a plurality of PPG signals (e.g., waveforms of the PPG signal) P1 to P7. According to an embodiment, the electronic device 200 may determine whether a PPG signal collected via the first sensor 211 has a peak of a threshold or more and has a similarity between periodic signals which is greater than or equal to a certain similarity level.

According to various embodiments, based on the first information with the designated quality is obtained (e.g., in response to determining the biometric signal represented by the first information has a signal quality equal to or greater than the designated signal quality), in operation 1130, the electronic device 200 (e.g., the processor 240) may provide a guide related to a blood pressure measurable situation. Additionally or alternatively, the electronic device 200 may perform a blood pressure measurement operation after providing the guide.

According to various embodiments, based on the first information with less than the designated quality is obtained (e.g., in response to determining the biometric signal represented by the first information has a signal quality less than the designated signal quality), in operation 1140, the electronic device 200 (e.g., the processor 240) may identify a measurement posture related to a subject based on second information collected via a second sensor 212. For example, the electronic device 200 may compare information obtained from the second sensor 212 with stored reference information to identify a measurement posture related to the subject.

According to various embodiments, in operation 1150, the electronic device 200 (e.g., the processor 240) may measure a temperature of the measurement area based on third information collected via a third sensor 213. The measurement area may be a part (e.g., a wrist) of the body adhering to (e.g., in contact with) the first sensor 211 in the state in which the electronic device 200 is worn on the body. According to an embodiment, the electronic device 200 may measure a temperature of a specific part (e.g., the measurement area) of the body, rather than a body temperature.

According to various embodiments, in operation 1160, the electronic device 200 (e.g., the processor 240) may provide a guide for inducing a measurable situation based on the measurement posture and the temperature of the measurement area. According to an embodiment, the electronic device 200 may provide at least one of text information and image information for inducing a posture of a user to a predetermined blood pressure measurement posture (and/or a position of a measurement device (e.g., the first sensor 211) to a predetermined measurement position) as a guide. For example, the electronic device 200 may provide at least some of the guides described with reference to FIGS. 6, 8, and 9.

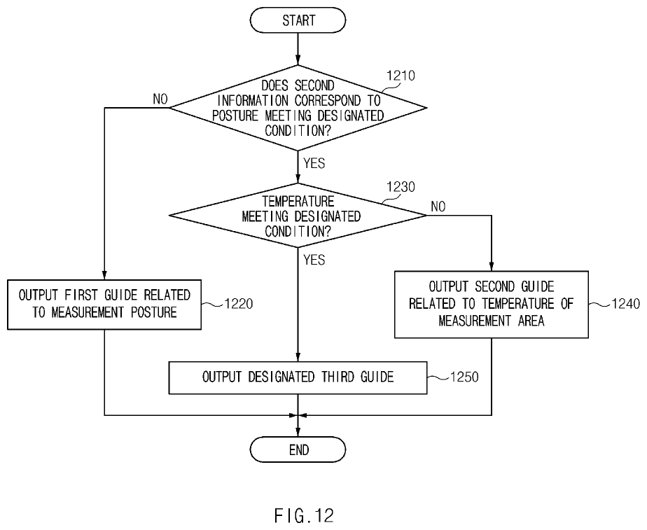

FIG. 12 is a flowchart illustrating an operation of providing a guide in an electronic device 200 according to various embodiments. Next, the respective operations in an embodiment below may be sequentially performed, but are not limited to being sequentially performed. For example, an order of the respective operations may be changed and at least two operations may be performed in parallel. Furthermore, at least one of the above-mentioned operations may be omitted according to an embodiment.

Referring to FIG. 12, in operation 1210, the electronic device 200 (e.g., a processor 240) according to various embodiments may determine whether second information corresponds to a posture meeting a designated condition. According to an embodiment, the electronic device 200 may determine whether the electronic device 200 is maintained according to a posture suitable for blood pressure measurement and/or a subject maintains a posture suitable for blood pressure measurement.

According to various embodiments, in response to determining the second information does not meet the designated condition (e.g., when the posture of the electronic device 200 and/or the subject remain(s) different from a measurement posture not suitable for blood pressure measurement), in operation 1220, the electronic device 200 (e.g., the processor 240) may output a first guide related to the measurement posture. According to an embodiment, the electronic device 200 may provide a guide for inducing the posture of the electronic device 200 and/or the posture of the subject to the predetermined blood pressure measurement posture. For example, the first guide may include the guide illustrated in 620 of FIGS. 6 and 830 of FIG. 8.

According to various embodiments, in response to determining the second information meets the designated condition (e.g., when the posture of the electronic device 200 and/or the subject remain(s) different from the measurement posture suitable for the blood pressure measurement), in operation 1230, the electronic device 200 (e.g., the processor 240) may determine whether third information corresponds to a temperature meeting the designated condition. According to an embodiment, the electronic device 200 may determine whether the temperature of the measurement area is a temperature suitable for blood pressure measurement.

According to various embodiments, in response to determining the third information corresponds to a temperature not meeting the designated condition (e.g., when the temperature of the measurement area is a temperature not suitable for blood pressure measurement), in operation 1240, the electronic device 200 (e.g., the processor 240) may output a second guide related to the temperature of the measurement area. According to an embodiment, the electronic device 200 may generate information (e.g., text information, image information, audio information) which guides a user in performing an appropriate action for increasing the temperature of the measurement area (e.g., Please rub the measurement area to raise the temperature.). For example, the second guide may include the guide illustrated in 810 of FIG. 8.

According to various embodiments, in response to determining the third information corresponds to the temperature meeting the designated condition (e.g., when the temperature of the measurement area is the temperature suitable for the blood pressure measurement), in operation 1250, the electronic device 200 (e.g., the processor 240) may output a designated third guide. According to an embodiment, the electronic device 200 may provide a blood pressure correction time point as the third guide. For example, the third guide may include the guide illustrated in 930 of FIG. 9.

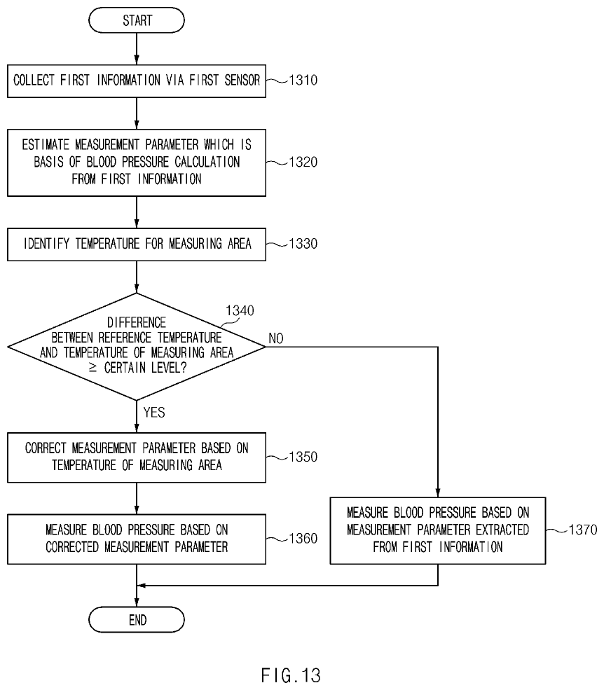

FIG. 13 is a flowchart illustrating an operation of an electronic device 200 according to various embodiments. Next, the respective operations in an embodiment below may be sequentially performed, but are not limited to being sequentially performed. For example, an order of the respective operations may be changed and at least two operations may be performed in parallel. Furthermore, at least one of the above-mentioned operations may be omitted according to an embodiment.

Referring to FIG. 13, in operation 1310, the electronic device 200 (e.g., a processor 240) according to various embodiments may collect (e.g., obtain) first information via a first sensor 211. According to an embodiment, the electronic device 200 may obtain the first information represented by a biometric signal (e.g., a PPG signal) for a measurement area (e.g., a wrist).

According to various embodiments, in operation 1320, the electronic device 200 (e.g., the processor 240) may estimate a value of a measurement parameter which is the basis of blood pressure calculation from the first information. According to an embodiment, the electronic device 200 may estimate a value of a measurement parameter, such as a cardiac output and/or total peripheral resistance (TPR), based on a feature point extracted from the PPG signal.

According to various embodiments, in operation 1330, the electronic device 200 (e.g., the processor 240) may identify a temperature of the measurement area. According to an embodiment, the electronic device 200 may measure the temperature of the measurement area based on third information collected via a third sensor 213. For example, in collecting the third information, the electronic device 200 may measure a temperature of a specific part (e.g., the measurement area) of the body, rather than a body temperature.

According to various embodiments, in operation 1340, the electronic device 200 (e.g., the processor 240) may determine whether a reference temperature and the temperature of the measurement area have a difference of a certain level or more. The reference temperature may be a temperature of the measurement area, which is stored at a blood pressure correction time point.

According to various embodiments, in response to determining the reference temperature and the temperature of the measurement area have the difference of the certain level or more (e.g., when the temperature of the measurement area is a temperature of a level at which a blood vessel constricts to the certain level or more), in operation 1350, the electronic device 200 (e.g., the processor 240) may correct the value of the measurement parameter based on the temperature of the measurement area. According to an embodiment, in correcting the value of the measurement parameter, the electronic device 200 may increase the value of the measurement parameter to the certain level. For example, the operation of correcting the value of the measurement parameter may refer to the description related to FIGS. 10A and 10B.

According to various embodiments, in response to determining the reference temperature and the temperature of the measurement area have the difference of the certain level or more (e.g., when the temperature of the measurement area is the temperature of the level at which the blood vessel constricts to the certain level or more), in operation 1360, the electronic device 200 (e.g., the processor 240) may provide a blood pressure measurement based on the corrected value of the measurement parameter.

According to various embodiments, in response to determining the reference temperature and the temperature of the measurement area do not have the difference of the certain level or more (e.g., when the temperature of the measurement area is not the temperature of the level at which the blood vessel constricts to the certain level or more), in operation 1370, the electronic device 200 (e.g., the processor 240) may provide a blood pressure measurement based on the measurement parameter extracted from the first information.

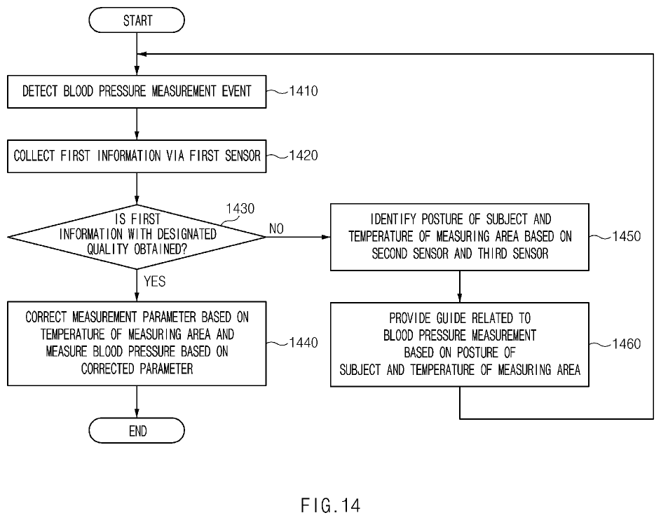

FIG. 14 is a flowchart illustrating an operation of an electronic device 200 according to various embodiments. Next, the respective operations in an embodiment below may be sequentially performed, but are not limited to being sequentially performed. For example, an order of the respective operations may be changed and at least two operations may be performed in parallel. Furthermore, at least one of the above-mentioned operations may be omitted according to an embodiment.

Referring to FIG. 14, in operation 1410, the electronic device 200 (e.g., a processor 240) according to various embodiments may detect a blood pressure measurement event. The blood pressure measurement event may be related to execution of a function of causing an operation of a first sensor 211.

According to various embodiments, in operation 1420, the electronic device 200 (e.g., the processor 240) may collect (e.g., obtain) first information via the first sensor 211. According to an embodiment, the electronic device 200 may obtain the first information represented by a biometric signal (e.g., a PPG signal) for a measurement area (e.g., a wrist).

According to various embodiments, in operation 1430, the electronic device 200 (e.g., the processor 240) may determine whether first information with designated quality is obtained. For example, the electronic device 200 may determine whether the biometric signal represented by the first information has a designated signal quality. As described with reference to 410 of FIG. 4, the first information with the designated quality (e.g., the biometric signal having the designated signal quality) may be some of a plurality of PPG signals (e.g., waveforms of the PPG signal) P1 to P7. According to an embodiment, the electronic device 200 may determine whether a PPG signal collected via the first sensor 211 has a peak of a threshold or more and has a similarity between periodic signals which is greater than or equal to a certain similarity level.

According to various embodiments, based on the first information with the designated quality is obtained (e.g., in response to determining the biometric signal represented by the first information has a signal quality equal to or greater than the designated signal quality), in operation 1440, the electronic device 200 (e.g., the processor 240) may correct a value of a measurement parameter estimated based on the first information based on a temperature of the measurement area and may provide a blood pressure measurement based on the corrected value of the measurement parameter. According to an embodiment, operation 1440 may include at least one of operations 1310 to 1370 of FIG. 13.

According to various embodiments, based on the first information with less than the designated quality is obtained (e.g., in response to determining the biometric signal represented by the first information has a signal quality less than the designated signal quality), in operation 1450, the electronic device 200 (e.g., the processor 240) may measure a posture of a subject and a temperature of the measurement area based on a second sensor 212 and a third sensor 213.

According to various embodiments, in operation 1460, the electronic device 200 (e.g., the processor 240) may provide a guide related to blood pressure measurement based on the posture of the subject and the temperature of the measurement area. According to an embodiment, operations 1450 and 1460 may include at least one of operations 1110 to 1160 of FIG. 11 and operations 1210 to 1250 of FIG. 12, which are described herein.

An electronic device 200 according to various embodiments may include a first sensor 211 which collects first information related to biometric information using a portion of a subject as a measurement area, a second sensor 212 which collects second information related to a posture of the electronic device 200, a third sensor 213 which collects third information related to a temperature of the measurement area, at least one processor 240, and a memory 230. The memory 230 may include at least one instruction.

According to an embodiment, the at least one instruction, when executed by the at least one processor 240, may cause the electronic device 200 to measure signal quality of a biometric signal represented by the first information and, in response to determining the signal quality is below a specified level, obtain a first result indicating whether the posture of the electronic device 200 corresponds to a designated reference posture based on the second information, obtain a second result indicating whether the temperature of the measurement area corresponds to a designated reference temperature based on the third information, and selectively provide a first guide related to the posture of the electronic device 200 or a second guide related to the temperature of the measurement area, based on the first result and the second result.

According to various embodiments, the second sensor 212 may include a sensor which collects information related to a wearing state of the electronic device 200. According to an embodiment, the instruction may cause the electronic device 200 to obtain a first additional result indicating whether the wearing state of the electronic device 200 corresponds to a designated wearing state (e.g., matches the designated wearing state within a certain degree of similarity) based on the second information and selectively provide the first guide, the second guide, or a first additional guide related to the wearing of the electronic device 200, based on the first result, the second result, and the first additional result.

According to various embodiments, the second sensor 212 may include a sensor which collects information related to a movement of the electronic device 200. According to an embodiment, the instruction may cause the electronic device 200 to obtain a second additional result indicating whether the movement of the electronic device 200 corresponds to a designated movement (e.g., matches the designated reference movement within a certain degree of similarity) based on the second information and selectively provide the first guide, the second guide, or a second additional guide related to the movement of the electronic device 200, based on the first result, the second result, and the second additional result.

According to an embodiment, the instruction may cause the electronic device 200 to provide the first guide related to the posture of the electronic device 200 based on the first result indicates the posture of the electronic device 200 does not correspond to the designated reference posture and provide another guide related to blood pressure measurement based on the temperature of the measurement area corresponding to the designated reference temperature is identified after providing the second guide related to the temperature of the measurement area.

According to an embodiment, the instruction may cause the electronic device 200 to provide the temperature of the measurement area together with the second guide.

According to various embodiments, the instruction may cause the electronic device 200 to, in response to determining the signal quality is above the specified level, estimate a value of a first measurement parameter related to blood pressure measurement based on the first information and provide a blood pressure measurement based on the value of the first measurement parameter.

According to an embodiment, the instruction may cause the electronic device 200 to obtain a second value of the first measurement parameter by correcting the value of the first measurement parameter based on the temperature of the measurement area and measure the blood pressure based on the second value of the first measurement parameter.

According to various embodiments, the first measurement parameter may include at least one of a cardiac output or total peripheral resistance (TPR).

According to various embodiments, the electronic device 200 may include a display 220. According to an embodiment, the instruction may cause the electronic device 200 to provide a visual guide through the display 220.

An electronic device 200 according to various embodiments may include a first sensor 211 (e.g., a biometric sensor) which collects biometric information using a portion of a subject as a measurement area, a third sensor 213 (e.g., a temperature sensor) which collects temperature information associated with the measurement area, at least one processor 240, and a memory 230. The memory 230 may include at least one instruction.

According to an embodiment, the at least one instruction, when executed by the at least one processor 240, may cause the electronic device 200 to estimate a value of a first measurement parameter related to blood pressure measurement based on the biometric information, measure a first temperature of the measurement area based on the temperature information, obtain a second value of the first measurement parameter by correcting the value of the first measurement parameter based on the first temperature and a second temperature stored in the electronic device 200, and provide a blood pressure measurement based on the second value of the first measurement parameter.

According to various embodiments, the first measurement parameter may include at least one of a cardiac output or total peripheral resistance (TPR).

An operating method of an electronic device 200 according to various embodiments may include collecting first information related to biometric information using a portion of a subject as a measurement area, measuring signal quality of a biometric signal represented by the first information, and, in response to determining the signal quality is below a specified level, obtaining second information related to a posture of the electronic device 200 and third information related to a temperature of the measurement area, obtaining a first result indicating whether the posture of the electronic device 200 corresponds to a designated reference posture (e.g., matches the designated reference posture within a certain degree of similarity) based on the second information, obtaining a second result indicating whether the temperature of the measurement area corresponds to a designated reference temperature (e.g., is equal to the designated reference temperature within a certain threshold difference) based on the third information, and selectively providing a first guide related to the posture of the electronic device 200 or a second guide related to the temperature of the measurement area, based on the first result and the second result.

According to various embodiments, the operating method of the electronic device 200 may include providing the first guide related to the posture of the electronic device 200 based on the first result indicates the posture of the electronic device 200 does not correspond to the designated reference posture.

According to various embodiments, the operating method of the electronic device 200 may include providing the second guide related to the temperature of the measurement area based on the first result indicates the posture of the electronic device 200 corresponds to the designated reference posture and the second result indicates the temperature of the measurement area does not correspond to the designated reference temperature.

According to various embodiments, the operating method of the electronic device 200 may include providing another guide related to blood pressure measurement when the temperature of the measurement area corresponding to the designated reference temperature is identified after providing the second guide related to the temperature of the measurement area.

According to various embodiments, the operating method of the electronic device 200 may include providing the temperature of the measurement area together with the second guide.

According to various embodiments, the operating method of the electronic device 200 may include, in response to determining the signal quality is above the specified level, estimate a value of a first measurement parameter related to blood pressure measurement based on the first information, obtaining a second value of the first measurement parameter by correcting the value of the first measurement parameter based on the temperature of the measurement area, and measuring a blood pressure based on the second value of the first measurement parameter.

According to various embodiments, the first measurement parameter may include at least one of a cardiac output or total peripheral resistance (TPR).

According to various embodiments, the operating method of the electronic device 200 may include collecting information related to at least one of a wearing state of the electronic device 200 and a movement of the electronic device 200 as at least a portion of the second information, obtaining at least one of a first additional result indicating whether the wearing state of the electronic device 200 corresponds to a specified wearing state based on the second information and a second additional result indicating whether the movement of the electronic device 200 corresponds to a specified movement, and selectively providing the first guide, the second guide, a first additional guide related to the wearing of the electronic device 200, or a second additional guide related to the movement of the electronic device 200, based on the first result, the second result, and the first additional result, and the second additional result.

A computer-readable storage medium according to various embodiments may store at least one instruction to collect first information related to biometric information using a portion of a subject as a measurement area, measure signal quality of a biometric signal represented by the first information, and, in response to determining the signal quality is below a specified level, obtain second information related to a posture of the electronic device 200 and third information related to a temperature of the measurement area, obtain a first result indicating whether the posture of the electronic device 200 corresponds to a designated reference posture based on the second information, obtain a second result indicating whether the temperature of the measurement area corresponds to a designated reference temperature based on the third information, and selectively provide a first guide related to the posture of the electronic device 200 or a second guide related to the temperature of the measurement area, based on the first result and the second result.

The electronic device according to various embodiments may be one of various types of electronic devices. The electronic devices may include, for example, a portable communication device (e.g., a smartphone), a computer device, a portable multimedia device, a portable medical device, a camera, a wearable device, or a home appliance. According to an embodiment of the disclosure, the electronic devices are not limited to those described herein.

It should be appreciated that various embodiments of the present disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include any one of, or all possible combinations of the items enumerated together in a corresponding one of the phrases. As used herein, such terms as “1st” and “2nd,” or “first” and “second” may be used to simply distinguish a corresponding component from another, and does not limit the components in other aspects (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used in connection with various embodiments of the disclosure, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program product may be traded as a product between a seller and a buyer. The computer program product may be distributed in the form of a machine-readable storage medium (e.g., compact disc read only memory (CD-ROM)), or be distributed (e.g., downloaded or uploaded) online via an application store (e.g., PlayStore™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities, and some of the multiple entities may be separately disposed in different components. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

What is claimed is:

1. An electronic device, comprising:
a first sensor which collects first information related to biometric information using a portion of a subject as a measurement area;
a second sensor which collects second information related to a posture of the electronic device;
a third sensor which collects third information related to a temperature of the measurement area;
at least one processor; and
a memory storing instructions that, when executed by the at least one processor, cause the electronic device to:
measure signal quality of a biometric signal represented by the first information; and
in response to determining the signal quality is below a specified level:
obtain, based on the second information, a first result indicating whether the posture of the electronic device corresponds to a designated reference posture;
obtain, based on the third information, a second result indicating whether the temperature of the measurement area corresponds to a designated reference temperature; and
selectively provide a first guide related to the posture of the electronic device or a second guide related to the temperature of the measurement area, based on the first result and the second result.

2. The electronic device of claim 1, wherein the second sensor comprises a sensor which collects information related to a wearing state of the electronic device, and
wherein the instructions cause the electronic device to:
obtain a first additional result indicating whether the wearing state of the electronic device corresponds to a designated reference wearing state, based on the second information; and
selectively provide the first guide, the second guide, and a first additional guide related to wearing the electronic device, based on the first result, the second result, and the first additional result.

3. The electronic device of claim 1, wherein the second sensor comprises a sensor which collects information related to a movement of the electronic device, and
wherein the instructions cause the electronic device to:
obtain a second additional result indicating whether the movement of the electronic device corresponds to a designated reference movement, based on the second information; and
selectively provide the first guide, the second guide, and a second additional guide related to the movement of the electronic device, based on the first result, the second result, and the second additional result.

4. The electronic device of claim 1, wherein the instructions cause the electronic device to:
provide the first guide related to the posture of the electronic device based on the first result indicates the posture of the electronic device does not correspond to the designated reference posture; and
provide the second guide related to the temperature of the measurement area based on the first result indicates the posture of the electronic device corresponds to the designated reference posture and the second result indicates the temperature of the measurement area does not correspond to the designated reference temperature.

5. The electronic device of claim 1, wherein the instructions cause the electronic device to:
identify that the temperature of the measurement area corresponds to the designated reference temperature, after providing the second guide; and
provide a third guide related to obtaining a blood pressure measurement, after identifying the temperature of the measurement area corresponds to the designated reference temperature.

6. The electronic device of claim 1, wherein the instructions cause the electronic device to provide the temperature of the measurement area together with the second guide.

7. The electronic device of claim 1, wherein the instructions cause the electronic device to, in response to determining the signal quality is above the specified level:
estimate a value of a first measurement parameter related to blood pressure measurement based on the first information; and
provide a blood pressure measurement based on the value of the first measurement parameter.

8. The electronic device of claim 7, wherein the instructions cause the electronic device to:
obtain a second value of the first measurement parameter by correcting the value of the first measurement parameter based on the temperature of the measurement area; and
measure the blood pressure based on the second value of the first measurement parameter.

9. The electronic device of claim 7, wherein the first measurement parameter comprises at least one of a cardiac output (CO) or total peripheral resistance (TPR).

10. The electronic device of claim 1, further comprising:
a display,
wherein the instructions cause the electronic device to selectively provide the first guide or the second guide through the display.

11. An electronic device, comprising:
a biometric sensor which collects biometric information using a portion of a subject as a measurement area;
a temperature sensor which collects temperature information associated with the measurement area;
at least one processor; and
a memory storing instructions that, when executed by the at least one processor, cause the electronic device to:
estimate a value of a first measurement parameter related to blood pressure measurement based on the biometric information;
measure a first temperature of the measurement area based on the temperature information;
obtain a second value of the first measurement parameter by correcting the value of the first measurement parameter based on the first temperature and a second temperature stored in the electronic device; and
provide a blood pressure measurement based on the second value of the first measurement parameter.

12. The electronic device of claim 11, wherein the first measurement parameter comprises at least one of a cardiac output (CO) or total peripheral resistance (TPR).

13. An operating method of an electronic device, the operating method comprising:
collecting first information related to biometric information using a portion of a subject as a measurement area;
measuring signal quality of a biometric signal represented by the first information; and
in response to determining the signal quality is below a specified level:
obtaining second information related to a posture of the electronic device and third information related to a temperature of the measurement area;
obtaining, based on the second information, a first result indicating whether the posture of the electronic device corresponds to a designated reference posture;
obtaining, based on the third information, a second result indicating whether the temperature of the measurement area corresponds to a designated reference temperature; and
selectively providing a first guide related to the posture of the electronic device or a second guide related to the temperature of the measurement area, based on the first result and the second result.

14. The operating method of claim 13, further comprising:
providing the first guide related to the posture of the electronic device based on the first result indicates the posture of the electronic device does not correspond to the designated reference posture; and
providing the second guide related to the temperature of the measurement area based on the first result indicates the posture of the electronic device corresponds to the designated reference posture and the second result indicates the temperature of the measurement area does not correspond to the designated reference temperature.

15. The operating method of claim 13, further comprising:
identifying that the temperature of the measurement area corresponds to the designated reference temperature, after providing the second guide; and
providing a third guide related to obtaining a blood pressure measurement after identifying the temperature of the measurement area corresponds to the designated reference temperature.

16. The operating method of claim 13, further comprising:
providing the temperature of the measurement area together with the second guide.

17. The operating method of claim 13, further comprising:
in response to determining the signal quality is above the specified level estimating a value of a first measurement parameter related to blood pressure measurement based on the first information; and
providing a blood pressure measurement based on the value of the first measurement parameter.

18. The operating method of claim 17, further comprising:
obtaining a second value of the first measurement parameter by correcting the value of the first measurement parameter based on the temperature of the measurement area; and
measuring the blood pressure based on the second value of the first measurement parameter.

19. The operating method of claim 17, wherein the first measurement parameter comprises at least one of a cardiac output (CO) or total peripheral resistance (TPR).

20. The operating method of claim 13, further comprising:
selectively providing the first guide or the second guide through a display of an electronic device.
